(19) United States
(12) Patent Application Publication
(10) Pub. No.: US 2010/0034935 A1
(43) Pub. Date: Feb. 11, 2010

(54) PRECISION TEMPERATURE COOKING SYSTEM AND METHOD

(75) Inventors: Sarah Wally, Waldwick, NJ (US); Juan M. Knight, Waldwick, NJ (US)

Correspondence Address: FOLEY & LARDNER LLP, 777 EAST WISCONSIN AVENUE, MILWAUKEE, WI, 53202-5306, US

(73) Assignee: Maverick Cuisine LLC

(21) Appl. No.: 12/512,913

(22) Filed: Jul. 30, 2009

Related U.S. Application Data
(60) Provisional application No. 61/087,415, filed on Aug. 8, 2008.

(51) Int. Cl. G01N 33/02; B65D 81/20

(52) U.S. Cl. 426/232; 99/472

(57) ABSTRACT

A precision temperature cooking system for cooking food products in a facility includes a vacuum packaging device to package the food products in vacuum-sealable bags, and a heated water bath or a heated water circulator to maintain a water bath at a cooking temperature that is substantially equal to a desired final temperature of a core portion of the food products, and a chilling/freezing system to blast-chill or flash-freeze the food products cooked by the water bath, and a refrigerated storage device to receive and store the frozen food products from the blast-chiller, and a high-temperature finishing component configured to impart a desired appearance to the outside of the cooked food product.

### CROSS REFERENCE TO RELATED APPLICATIONS

The present Application claims the benefit of priority of co-pending U.S. Provisional Patent Application No. 61/087,415, titled “Low Temperature Cooking System and Method” filed on Aug. 8, 2008, the disclosure of which is hereby incorporated by reference in its entirety.

### FIELD

The present invention relates generally to a precision temperature cooking system and method. The present invention relates more particularly to a precision temperature cooking system, and a method for designing a precision temperature cooking system to replace existing high temperature cooking systems in commercial, industrial and institutional facilities.

### BACKGROUND

This section is intended to provide a background or context to the invention recited in the claims. The description herein may include concepts that could be pursued, but are not necessarily ones that have been previously conceived or pursued. Therefore, unless otherwise indicated herein, what is described in this section is not prior art to the description and claims in this application and is not admitted to be prior art by inclusion in this section.

Conventional “high” temperature cooking systems are well known and typically involve heating an article such as a food product at a relatively high temperature until the internal (core) temperature reached a desired level. However, such high temperature cooking systems and equipment have a number of disadvantages, such as (for example) resulting in outer portions or layers of the food product being overcooked due to the temperature gradient between the cooking device and the desired internal temperature of the food product, and loss of yield (i.e. shrinkage) of the food product due to the high temperatures, increased waste of the food product due to even slight errors in judging the timing and temperature needed to obtain the desired final internal temperature of the food product, loss of flavor due to vapors and juices being driven from the food product by the high cooking temperatures, high energy consumption required to heat the high temperature cooking equipment, the necessity for large and energy intensive ventilation systems to remove aromas, gases and vapors that are emitted from the food products and the heat generated by the cooking devices, the need to cook the food article at approximately the time of food service to prevent the food from cooling, or from drying out (due to prolonged air exposure or from storage and reheating), which typically results in a cyclic and labor-intensive pattern of cooking large quantities of food articles in short periods of time at frequencies dictated by scheduled food service times, and high levels of chef/staff presence to accommodate the demand to cook the food products at approximately the time of food service, and high levels of skill, expertise and training in cooking the food products with high temperature devices in an attempt to minimize errors and waste associated with the precision necessary to simultaneously obtain a specific internal temperature of many food products using high temperature cooking devices.

Precision temperature cooking systems are intended to minimize or eliminate the disadvantages of high temperature cooking systems. Some relatively primitive methods of low temperature cooking are generally known, such as wrapping food products in a protective coating and poaching in boiling (or near boiling) water for a sufficient period of time necessary to obtain the desired internal temperature of the food product, however, such known “low” temperature cooking systems still result in a relatively substantial temperature gradient between the desired internal temperature of the food product and the heat source, and tend to have at least some of the disadvantages of the high temperature systems.

Thus there is a need to provide a precision temperature cooking system that overcomes the disadvantages of the high temperature cooking systems and the known low temperature cooking systems. There is also a need for a method of operating such precision temperature cooking systems. There is also a need to provide a method of designing a precision temperature cooking system for use in new applications, and for replacing high temperature systems in a wide variety of existing applications.

Accordingly, it would be desirable to provide a precision temperature cooking system that minimizes a temperature gradient between a heat source and the desired internal temperature of the food product(s). It would also be desirable to provide a method of operating such as precision temperature cooking system, and a method of designing a precision temperature cooking system for new applications and for replacing existing high temperature cooking systems.

### SUMMARY

One embodiment of the present invention relates to a precision temperature cooking system for food products. The system includes a vacuum packaging device to package the food products in vacuum-sealable bags, and a heated water bath or a heated water circulator to maintain a water bath at a cooking temperature that is substantially equal to a desired final temperature of a core portion of the food products. A blast-chiller or other chilling method chills or freezes the food products cooked by the water bath and a refrigerated storage device receives and stores the frozen food products from the blast-chiller. A high-temperature finishing component imparts a desired appearance to the outside of the food product.

Another embodiment of the present invention relates to a method of operating a precision temperature cooking system for cooking food products in a facility. The method includes the following steps. Identifying a scope of food products to be served by the facility. Obtaining and preparing the food products to be served in the facility by sourcing the food products, providing separate areas on a work surface for raw food products and cooked food products, and cleaning the food products. Vacuum packing the food products with any other desired ingredients, labeling the food products with a packaging date, an expiration date, and an identification of the food product. Blast-chilling the food products to a desired storage temperature and then transferring the food products to a refrigerated storage device. Establishing minimum cooking times and minimum cooking temperatures for the food products. Storing the minimum cooking times and minimum cooking temperatures in a database. Cooking the food products in a precision temperature cooking component operating at a temperature substantially equal to a desired final temperature of a core portion of the food product. Receiving instructions to provide the food products for food service delivery. Regenerating the food product in the lower temperature cooking component, and finishing the food product on a high temperature cooking component to provide a desired surface condition on the food product.

A further embodiment of the present invention relates to a method of providing a precision temperature cooking system for cooking food products in a facility. The method includes the following steps. Analyzing existing operations of the facility, which includes analyzing the food products offered by the facility, where the food products are defined by one or more menus, determining a representative and maximum volume and frequency that the food products are served by the facility, determining a representative cost of the food products served at the facility, analyzing a scope and location of existing high temperature cooking components used in the facility to cook the food products and a location and a capacity of any existing ventilation devices, determining a representative amount of energy required by the existing high temperature cooking components to cook the food products, determining a representative amount of existing labor necessary to cook the food products with the existing high temperature cooking components, and determining an amount of existing space required for processing the food products with the existing high temperature cooking components and with the amount of existing labor. A next step includes designing the precision temperature cooking system by evaluating the food products to be served in the facility, which includes identifying precision temperature cooking components for cooking the food products, determining a size and a quantity of the precision temperature cooking components necessary to cook a quantity of food products sufficient to meet the maximum volume and frequency that the food products are served by the facility, identifying one or more high temperature finishing components for use in finishing the food products cooked by the precision temperature cooking components, determining a size and a quantity of blast-chiller devices sufficient to blast-chill or flash-freeze a surplus quantity of the food products for subsequent regenerating, determining a size of refrigerated storage space sufficient to maintain the surplus quantity of the food products in a chilled or frozen state, establishing a first layout of the precision temperature cooking components to fit within the existing space, and establishing a second layout of the finishing components to fit within the existing space and proximate the existing ventilation device. A next step includes quantifying a savings obtainable from use of the precision temperature cooking components to cook the food products, which includes calculating a labor cost savings from reduction in labor resulting from cooking the food products with the precision temperature cooking components and normalizing high demand periods and low demand periods for service of the food products in the facility, and calculating an energy savings resulting from cooking the food products with the precision temperature cooking components.

### BRIEF DESCRIPTION OF THE DRAWINGS

FIG. 1 is a schematic representation of a precision temperature cooking system according to one embodiment.

FIG. 2 is a schematic representation of a precision temperature cooking system according to another embodiment.

FIG. 3 is a schematic representation of an ingredient control center for a precision temperature cooking system according to an exemplary embodiment.

FIG. 4 is a schematic representation of a precision temperature kitchen having a precision temperature cooking system according to an exemplary embodiment.

FIG. 5 is a block diagram of a method of operating a precision temperature cooking system according to another embodiment.

FIGS. 6A-6E are block diagrams of a method of designing a precision temperature cooking system according to another embodiment.

### DETAILED DESCRIPTION

According to the illustrated embodiments, a precision temperature cooking system and method of designing and operating a precision temperature cooking system are shown for use in a wide variety of applications, such as a kitchen (or any other suitable food preparation area, whether stationary or mobile) associated with facilities such as an educational facility (e.g. school, college, university, etc.), a healthcare facility (e.g. hospital, hospice, extended-care facility, etc.), commercial kitchen (e.g. fine-dining restaurants, multi-unit restaurant operations, take-out and catering service, mobile vendors, caterers, etc.), hotel production and room service, military kitchens, kiosks and other quick-food applications, etc.

The precision temperature cooking system according to the exemplary embodiments involves food products (e.g. meat, fish, vegetables, etc.) and other related ingredients (e.g. spices, seasonings, sauces, etc.) being vacuum packed in a vacuum sealable bag and then slowly cooked in a heated water bath at a controlled “low” temperature specifically adapted for the particular food product. The exclusion of air in the vacuum bag is intended to reduce the growth of aerobic bacteria, and delays any spoilage of the food product or other ingredients. Cooking the food products at lower temperatures for extended periods of time is intended to provide a wide range of benefits, in comparison to high temperature cooking systems. Including, for example, minimal loss of moisture and weight, preservation of flavor and aroma as water soluble substances are retained, enhancement of flavors, retention of colors and reduction in a need for salt, preservation of nutrients as water-soluble minerals are not leached. Cooking in a vacuum bag also eliminates any products being placed directly into the water and enhances retention of vitamins (as opposed to high temperature cooking operations such as steaming and boiling of the food products), requires little additional fat (if any) during precision temperature cooking, and improves the consistency of results for repeated cooking of such food products.

According to the exemplary embodiments of the precision temperature cooking system, the food products are cooked using specific cooking times, and predefined and accurately controlled temperatures, and specially designed precision temperature cooking components. The precision temperature cooking components include vacuum packing machines, heated water baths and heated water circulators, combined temperature and steam ovens (e.g. “combi-oven”), a chilling device, such as a blast-chiller (e.g. flash-freezer), a refrigerated storage device, and a high temperature finishing device.

The precision temperature cooking system of the exemplary embodiments is intended to provide a number of operational benefits, including (among others) the following. The ability to maximize advance preparation of food products by cooking and then chilling quickly in the blast-chiller or using an equivalent chilling method after the cooking bath process has finished, so that the food products can be safely stored until needed, and then “regenerated” in the heated bath and “finished”. The ability to minimize waste by advance preparation of controlled portions. Minimum shrinkage of contents during the cooking process, typically from about 30% (which is typical for high temperature cooking systems) to less than 5% in most cases and thus results in greater yields of the food product. Less expensive (e.g. cheaper) cuts of meat can be used as the precision temperature cooking system improves tenderness of the food products. Extra (e.g. unanticipated) demand for the food products can be drawn from cold storage. Less energy consumption is required by the precision temperature cooking components, as compared to high temperature cooking system components. Elimination or reduction of gas-fired cooking components tends to reduce ambient temperatures in the kitchen and fire risk. Several meals from “starter to dessert” can be regenerated simultaneously in the same heated bath device, thus reducing cleanup time. With minimal training, relatively unskilled staff can operate the components of the precision temperature cooking system because reduction of a temperature gradient between the cooking component and the desired final temperature of the food product requires less skill and attention. The ability to simplify food service activities is particularly effective for applications such as room service during the night hours, when maintaining a staff of personnel and high temperature cooking components in standby is not cost-effective. Further, work planning, preparation and cooking outside of service times is improved, such as planning ahead for banqueting and other large-scale food service activities. The precision temperature cooking system according to the embodiments described herein is also intended for use in applications requiring six sigma precision, due to the ability of the system to accurately control the cooking temperature and the finished internal temperature of the food product. For example, conventional cooking equipment typically operates with a temperature variation of ±20° F. (or more) above or below the setpoint, and spatial variation within the cooking device itself, thus often requiring kitchen personnel to set their cooking temperature well above the final desired temperature for the food product. For instance, when the desired food product internal temperature is 165° F., the actual final is typically about 185° F. or more to account for the uncertainty in actual cooking temperature. Further, when large quantities of food are being cooked, usually probes are inserted into only one or two of the food products and are used as a representative indication of the internal temperature of all the food products being cooked, thus often leading to a large quantity of food that is often under-cooked or over-cooked. The system according to the exemplary embodiments described herein overcomes these deficiencies of the conventional cooking systems by providing a precision cooking systems and equipment with a temperature variation typically on the order of approximately ±1° F. temperature variation above or below the setpoint, and spatial variation within the cooking device, thus permitting control of the cooking temperature with much greater precision, such as that required by facilities having six sigma requirements or programs. The resulting food products are uniformly cooked to an internal temperature that is much closer to the actual required temperature (e.g. 166-168° F. for food product applications with a temperature requirement of 165° F.).

The Applicants believe that the precision temperature cooking system described in the exemplary embodiments will lead to kitchens of the future that are dominated by advanced technology, smaller culinary staff, 24 hour customer demand for restaurant-quality food, and shrinking kitchen space (e.g. footprints, etc.), and remotely-generated computer-assisted instructions for menu, recipe and/or dietary instructions or guidelines for use with the precision temperature cooking system. Kitchens and other food service environments will be able to take advantage of multi-functional equipment that delivers more with less space, and the ability to arrange precision temperature cooking systems vertically with an equipment platform (e.g. structure, suite, etc.), rather than the typical horizontal arrangement of most high temperature cooking systems, will further enhance space utilization. The precision temperature cooking system is intended to permit such kitchens of the future to offer high quality 24-hour menus and service to provide for rapid-response, late night dining and room service with outstanding product and minimal staffing requirements. Advances in cooking technology tend to mean fewer people are needed to conduct the same amount of work, leading to labor savings from smaller kitchen staffs. Commercial food service is generally made up from the elements of cooking equipment, food products and labor (e.g. staff, people, etc.). The precision temperature cooking systems tends to improve control of the food service by minimizing the uncertainties associated with the labor element through a system of precision temperature cooking components that is easy to operate, permits increased usage of food products packaged/prepared offsite and cooked onsite to provide on-demand, high volume, high-quality results consistently. Menus, recipes and dietary information (e.g. special requirements, restrictions, etc) can be generated at one location (e.g. by dieticians, executive chefs, etc.) and transmitted directly to the kitchen at another location for use by the staff in operating the components of the precision temperature cooking system to deliver the desired food service. Further, the components of the precision temperature cooking system may be programmed to receive such instructions directly (e.g. for cooking time, temperature, etc.) to further enhance quality and reduce the variability of the kitchen staff's influence on the food service results.

Accordingly, all such concepts and variations of the precision temperature cooking system, method for operating a precision temperature cooking system and method of designing a precision temperature cooking system are intended to be within the scope of this disclosure.

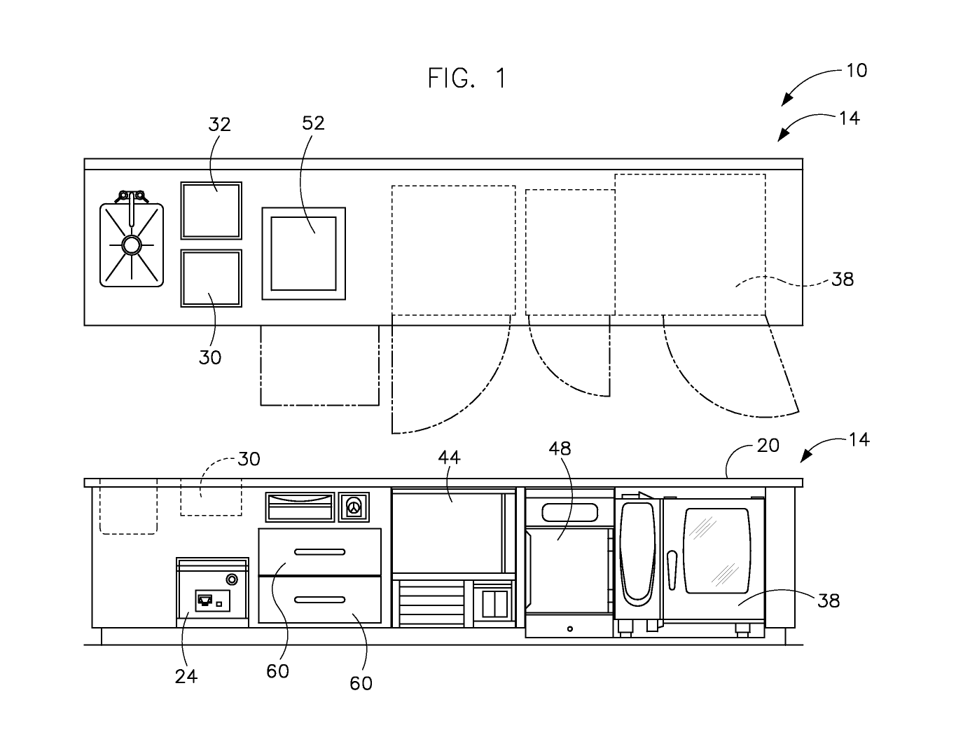

Referring to FIG. 1, a precision temperature cooking system 10 is shown according to an exemplary embodiment. System 10 is shown to include a combination of precision temperature cooking components packaged in a precision temperature cooking suite 14 (structure, housing, platform, cabinet, etc.). Suite 14 comprises an enclosure intended to hold all of the components in a space-saving, self-contained orientation, and in a sequence that enhances workflow of the precision temperature cooking process. Suite 14 may be formed from any suitable material intended to withstand the rigors of a high-demand kitchen environment, such as stainless steel, and may be internally pre-wired and pre-plumbed to all components to provide a single external plumbing and wiring connection to facilitate installation at a facility. Suite 14 also includes a work surface 20 that may include any one of a wide variety of suitable materials, such as a solid surface material (e.g. Corian®, Avonite®, etc.), or a wood material such as a maple or the like, stainless steel, or other suitable material.

Referring further to FIG. 1, the precision temperature cooking components are shown to include a vacuum packaging machine 24, one or more heated water bathes 30 and heated water circulators 32, a combination thermal-steam oven 38 (“combi-oven”), a blast-chiller 44, a refrigerated storage compartment 48, a finishing device 52, and one or more warmer drawers 60.

The vacuum packaging machine 24 is provided for use in vacuum packaging the food product(s) along with any other desired ingredients inside a plastic bag. Plastic bags of various sizes and thicknesses may be used. Generally, the maximum width of the bag is dependent upon on the width of a seal bar on the particular vacuum packaging machine. There are two primary types of vacuum packaging machines: chamber-type and external-type (such as a “food saver” type device). According to one embodiment, smooth surfaced bags, having a thickness within the range of approximately 20 to 30 mil are used in chamber-type vacuum packaging machines, and bags with one surface having a reticulated pattern pressed in are used for external-type machines. According to another embodiment, heat-shrinkable bags may be used for food products that are intended to retain their original shape, and are designed to shrink comfortably around the food product when the bag is heated.

In chamber-type machines, the food product is placed in the plastic bag, and then placed inside the chamber with the open end of the bag extending across the seal bar. The chamber is closed, and a vacuum pump removes the air within the chamber. The vacuum packaging machine may be programmed to draw a vacuum for a fixed amount of time, or to obtain a percentage vacuum (e.g. 99% to 99.5% or other suitable vacuum level). When the desired vacuum level has been obtained, the seal bar presses down on the bag and applies heat to seal the bag. The chamber is then opened, and the bag contracts, as its internal pressure is at the vacuum level attained relative to atmosphere. According to one embodiment, the vacuum packaging device 24 is a chamber-type machine such as are commercially available from Minipack®-America. In exterior or “food saver” type machines, the food product is bagged, and then the open end of the bag is placed in the machine. The top is lowered, and the vacuum is applied. At the end of the vacuuming process, the seal bar heats up and seals the bag. These machines are typically lower cost than the chamber type, but may not obtain as high a vacuum level. One advantage of the exterior-type is that the bag length is generally not limited by chamber size, (e.g. if one wanted to vacuum package a whole fish, the fish may be placed in a suitably long bag, and the open end of the bag is placed in the vacuum machine. According to one embodiment, the vacuum packaging device 24 is an external-type machine such as are commercially available from Minipack®-America. According to an alternative embodiment, the vacuum packaging machine may have a reversible seal bar, intended to allow for outside the chamber packaging using the correct type of bag.

Referring further to FIG. 1, the heated water baths 30 and heated water circulators 32 are components operable to provide a precision temperature heat source for cooking (or regenerating) the vacuum-packaged food products. Operation of the heated water baths 30 and heated water circulators 32 differs from previous “low” temperature systems by reducing the cooking temperature of the water bath to approximately the same temperature as the desired final temperature of the food product. Previously, when cooking was done in a water bath (e.g. poaching, boiling, cooking in a double-boiler or bain-marie, etc.), the water temperature was generally set higher than the final desired temperature of the cooked food product. For example, if one were cooking a steak to medium rare at 60° C. (about 140° F.), the water bath (or bain-marie) was usually set at approximately 90° C. (about 195° F.). A temperature probe would be placed through the bag into the center of the food product, which was cooked until the probe indicated that the center was at the desired temperature. However, for the center of the food product to reach the proper temperature, the outside of the food product would be at a temperature approaching (or equaling) the temperature of the bath, which represents the temperature gradient between the bath temperature and the temperature of the food through to the center of the product. In the steak example, only the center would be medium rare, while the outside would be overdone.

The heated water bath 30 of the precision temperature cooking system operates at approximately the same temperature as the desired finished cooking temperature of the food product. The cooking time in the water bath will be longer than in previous cooking systems, but the result will be a substantially uniform temperature through the food product, because there will be no (or relatively little) temperature gradient between the center of the food product and the temperature of the water bath when cooking of the food product is completed. In the steak example, if the temperature of the bath is set for medium-rare (e.g. about 60° C.), then the steak will be medium-rare from the outside to the center. The more accurately the temperature of the water bath can be controlled and maintained, the more uniform the temperature throughout the food product will be.

Generally, the heated water baths 30 are better suited to food products that tend to have a longer cooking time, and heated water circulators 32 are better suited for use with food products having a relatively short cooking time. The heated water bath 30 includes a stainless steel pan with a heating element (e.g. heated blankets, etc.) wrapped around the pan for transferring heat to the water bath, a temperature sensor and a control panel. According to one embodiment, a size of the heated water bath is within a range of approximately 2 quarts to 30 quarts of water, with the capability for temperature uniformity within a range of approximately ±1° F.), and includes a cover to help maintain temperature of the bath and to reduce evaporation. According to one embodiment, the heated water bath 30 is of a type commercially available from Julabo USA of Allentown, Pa.

The heated water circulator 32 is a relatively compact device that includes a motor and control panel on an upper portion, and a heating element, temperature sensor and impeller on a lower portion, and is adapted for use with a separate container. The lower portion is immersed in the water, and the desired temperature is set on the control panel. The circulator may be fixed to the side of a container of water (e.g. pots and pans or the like), usually with a depth of at least about 6 inches. The container is generally sized for a volume and surface of water that the power of the circulator can handle, to insure the desired uniformity of water temperature within the container is obtained. According to one embodiment, the heated water circulator 32 is of a type commercially available from Julabo USA of Allentown, Pa. According to alternative embodiments, the heated water circulator may be provided with a specially designed water bath container affixed to the circulator.

The Applicants have found that the heated water bath 30 is accurate enough for most food products. However, an initial temperature transient typically occurs at the beginning of the cooking cycle (when the relatively cold food products are immersed in the heated water bath), in which the heated water circulator 32 tends to provide better heat transfer and shorter recovery time. For a long cook time product (e.g. 48 hours for short ribs or the like), the initial temperature transient and recovery time is not a significant factor. However, for a short cook time (e.g. 5 minutes for a fish serving or the like), the initial temperature transient may be a more significant factor, and the heated water circulator 32 may be the preferred source of heat for precision temperature cooking. Accordingly, the heated water bath 30 and the heated water circulator 32 may be adapted for various food products depending upon factors such as the cooking time duration, the power (e.g. wattage) of the heating element and its ability to recover the initial temperature transient, the number and frequency of initial temperature transients (i.e. placement of the food products in the water), etc.

Referring further to FIG. 1, the thermal-steam oven(s) (“combi-oven”) 38 is provided as another heat source for precision temperature cooking (or regenerating) both non-packaged and vacuum-packaged food products. Combi-oven 38 is intended to provide a precision-temperature steam and heat source for cooking food products that are not ideally suited for submersion in a heated water bath, such as (for example) delicate items, bakery products, certain vegetables, fish, etc. According to one embodiment, the combi-oven 38 is of a type commercially available from Rational Cooking Systems of Schaumburg, Ill., or of a type know as CVap and commercially available from Winston Industries in Louisville, Ky. The combi-oven 38 is capable of operating in a moist heat (steam) mode (such as for low temperature cooking), a dry heat mode (such as for use as a high temperature finishing device for “finishing” the surface of a food product cooked using one of the precision temperature devices, and a combination mode.

Referring further to FIG. 1, a blast-chiller 44 for flash-freezing food products cooked using either of the precision temperature cooking components (e.g. heated water bath 30, circulating water bath 32, combi-oven 38, etc.) is provided according to an exemplary embodiment. Blast-chiller 44 is intended to flash freeze or otherwise quickly chill the food products for storage in the refrigerated storage location 48. Flash-freezing is advantageous (for example) so that large quantities of food products can be prepared and cooked in advance and later quickly regenerated (re-thermalized, reheated, etc.) when food service of large quantities of the food products are desired (so as to help normalize the often cyclical nature of the kitchen preparation and cooking activities), or as a readily available supply of food products in the event a greater quantity is required then was anticipated, or for applications where high quality food is required on demand and on short notice during off-normal times (e.g. 24 hour room service, etc.). According to one embodiment, the blast-chiller 44 is of a type commercially available from Irinox USA of North Easton, Mass.

Referring further to FIG. 1, a refrigerated storage device 48 is provided for use in storing food products that have been flash-frozen or blast-chilled, until such products are retrieved and regenerated for service in the facility. Refrigerated storage device 48 may operate at any desired storage temperature, such as minus (−)20° F. to (+)20° F. or other suitable temperature for a particular type of food product. According to alternative embodiments, the refrigerated storage device may be provided separately from the suite (or in addition to the suite), such as a walk-in cooler, walk-in freezer, etc.

Referring further to FIG. 1, the warmer drawers 60 are provided for use in temporarily storing food products that have been cooked and/or regenerated by the precision temperature cooking components and are awaiting finishing and/or service to the facility. Although two warmer drawers 60 have been shown by way of example, any number of warmer drawers may be provided, and operating at a common temperature or different temperatures.

Referring further to FIG. 1, a finishing device 52 is shown according to an exemplary embodiment. Finishing device 52 is intended to provide a high-temperature device capable of providing a relatively quick finishing “touch” on the food product that has already been cooked with the precision temperature cooking components, to provide an attractive, aesthetically pleasing or other desirable appearance on the food product. For example, if the food product is a type of meat, the surface of the meat may be finished to have an appearance that is browned, charred, seared, grilled, or the like. However, other food products may be finished having any other desirable surface treatment or preparation intended to impart an aesthetically and culinary pleasing appearance. The finishing device 52 is shown by way of example to include a plancha, but may be any one or more of a variety of other high temperature devices, such as a grill, griddle, burner, rotisserie, dry heat mode of the combi-oven, etc.

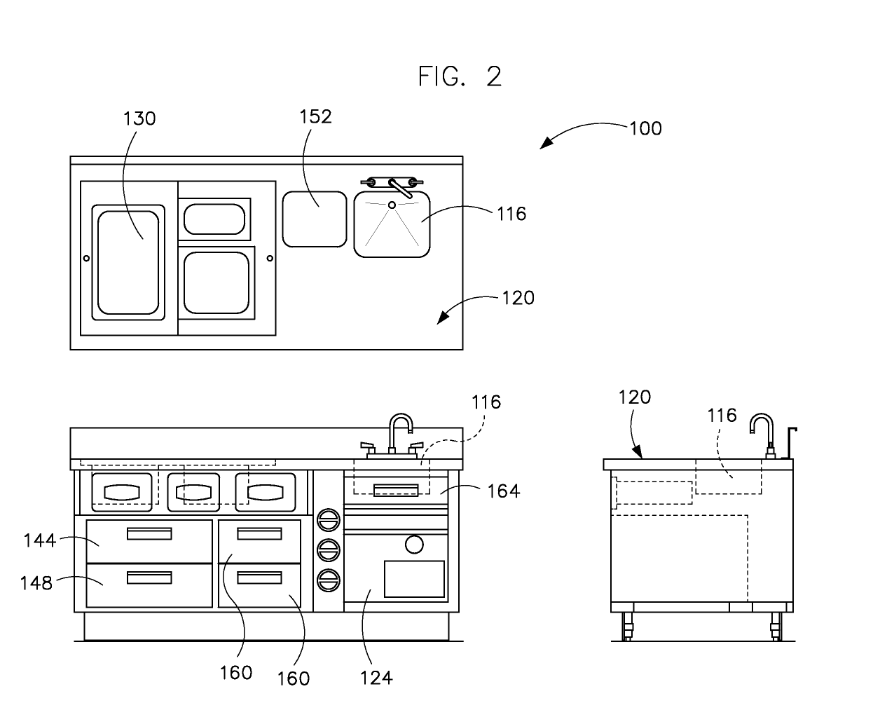

Referring now to FIG. 2, a precision temperature cooking system 100 is shown according to another exemplary embodiment. System 100 is shown to include a combination of precision temperature cooking components packaged in a precision temperature cooking suite 114. Suite 114 comprises an enclosure intended to hold all of the components in space-saving, self-contained orientation, and in a sequence that enhances workflow of the precision temperature cooking process. Suite 114 may be formed from any suitable material intended to withstand the rigors of a high-demand kitchen environment, such as stainless steel, and may be internally pre-wired and pre-plumbed to all components to provide a single external plumbing and wiring connection to facilitate installation at a facility. Suite 114 also includes a work surface that may include any one of a wide variety of suitable materials, such as a solid surface material (e.g. Corian®, Avonite®, etc.), or a wood material such as a maple or the like, stainless steel, or other suitable material.

The precision temperature cooking components are shown to include a preparation sink 116, a work surface 120, a vacuum packaging machine 124 (provided by way of example as a pullout drawer), drop-in heated water baths 130 in three sizes, a blast-chiller 144 (provided by way of example as a pullout drawer), a refrigerated storage compartment 148 (provided by way of example as a pullout drawer), a finishing device 152 shown for example as a plancha, a warmer cabinet 160, and a utility drawer 164.

The precision temperature cooking components according to the exemplary embodiment illustrated in FIG. 2 are arranged in a manner to more efficiently support kitchen labor and workflow. For example, according to the illustrated embodiment, the components are arranged in the following manner. The sink 16 is provided adjacent to the work surface 120 for preparation of the food products and bagging the prepared food products in vacuum sealable bags. The “bagged” food products may then be sealed in the vacuum packaging machine 124 shown disposed beneath the work surface 120. The vacuum packaged food products may then be cooked in an appropriate one of the drop-in heated water baths 130 (each of which may be operating at a common temperature or different temperatures, according to the specific food products to be cooked). Once the food products are cooked, they may be placed directly in the blast-chiller 144 disposed directly beneath the heated water baths 130 for rapidly freezing the food products and then transferred to the refrigerated storage compartment 148 directly adjacent to the blast chiller 144 for holding until a later time as desired/required to accommodate food service schedules and needs of the facility. When the cooked/stored food products are desired for food service, they may be withdrawn from refrigerated storage 148 and placed in the heated water bath 130 to regenerate the food product to the desired temperature, and then removed from the vacuum sealable bags on the work surface 120 and transferred directly to the finishing device 152 for finishing the surface of the food product, and then placed in the warmer drawer 160 (as needed) until required for food service delivery. Alternatively, when chilled storage of the cooked food products is not required, the food products may be removed from the heated water baths 130 and removed from their bags on the work surface 120, and then transferred directly to finishing device 152 for finishing the surface of the food product and then temporarily stored in warmer drawer 160, or delivered directly for food service.

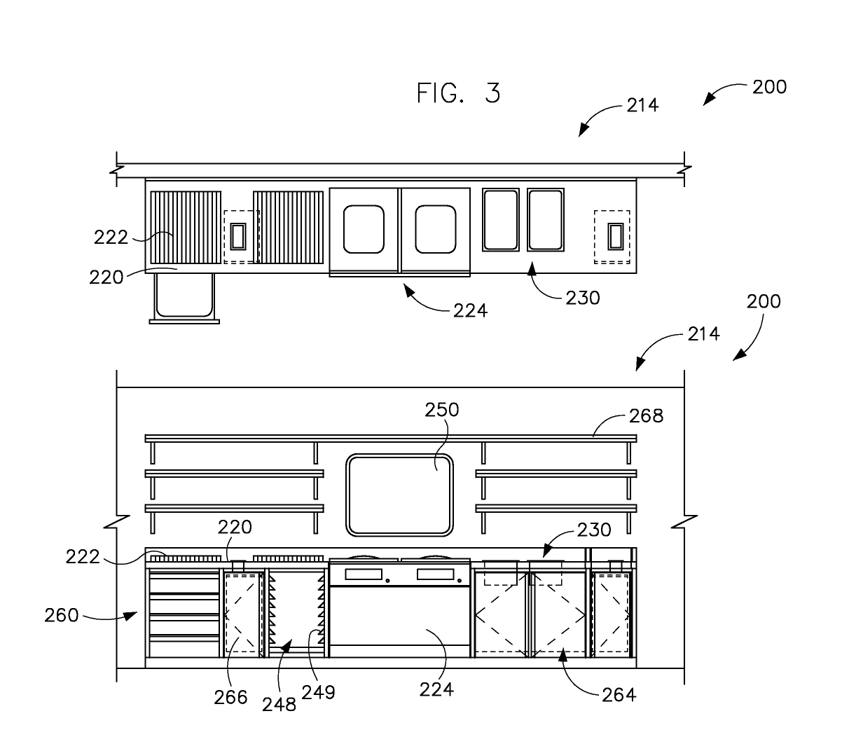

Referring now to FIG. 3, an ingredient control center 212 for a precision temperature cooking system 200 is shown according to another exemplary embodiment. Ingredient control center 212 is intended to provide a designated location for receiving (e.g. from storage or supplier, etc.), ingredient control, preparing, vacuum packaging and labeling food products having any of a wide variety of particular ingredients for intended applications. Such applications may be related to specialized dietary restrictions such as for use in hospitals and healthcare facilities, or for a wide variety of specialized menu offerings in a commercial food service facility, etc. The ingredients may be suitable ingredients in addition to the food products (e.g. seasonings, spices, sauces, etc.). The ingredients are applied to the food products (or otherwise included with the food products in a vacuum packaging bag) and then sealed in the bag and using a vacuum packaging machine and labeled to identify the prepared/seasoned vacuum packaged food products with a packaging date, an expiration date, cooking time and temperature, and an identification of the contents (e.g. food product, type of other ingredients, dietary restriction notations (low/no sodium, etc.), intended food service event, intended recipient, etc.). The ingredient control center may be provided with suitable software on an appropriate computing device to specify the size of the food portions (e.g. by weight, etc.) and the type and amount of other ingredients to be included with the food products, and to control the temperature of the water baths and precision-temperature cooking duration required for the particular packaged food product, and to provide pre-printed labels to accurately represent the specific information related to the packaged food product.

The ingredient control center is shown to include a combination of precision temperature cooking components packaged in a precision temperature cooking suite 214 intended to centralize all the equipment necessary for establishing ingredient control in the assembly and packaging of the food products with the desired ingredients into vacuum-sealed packages with identification labels that are pre-cooked in the precision-temperature cooking equipment. Suite 214 comprises an enclosure intended to hold all of the components in space-saving, self-contained orientation, and in a sequence that enhances workflow of the ingredient control portion of the precision temperature cooking process. Suite 214 may be formed from any suitable material intended to withstand the rigors of a high-demand kitchen environment, such as stainless steel, and may be internally pre-wired and pre-plumbed to all components to provide a single external plumbing and wiring connection to facilitate installation at a facility. Suite 214 also includes a work surface 220 that may include any one of a wide variety of suitable materials, such as a solid surface material (e.g. stainless steel, Corian®, Avonite®, etc.), and may include cutting boards 222 formed from a suitable material (e.g. a wood material such as a maple or the like), or other suitable material.

The components of the ingredient control center of the precision temperature cooking system are also shown to include a vacuum packaging machine 224 (shown as two vacuum packaging machines and provided by way of example as a pullout drawer); drop-in heated water baths 230 in various sizes and operating temperatures; a refrigerated storage compartment 248 (shown for example with storage racks 249); a user interface 250 (e.g. touch screen display or other suitable user interface) for a computing device operated by software that provides the desired ingredient control and cooking information for the food products; warmer drawers 260; utility cabinet 264; a drop-in, pull-out waste receptacle 266, and storage shelves 268. Other suitable components may be provided with the ingredient control center portion of the precision temperature cooking system as may be desirable to establish an efficient station suited for a desired application.

The ingredient control center or station of the precision temperature cooking components according to the exemplary embodiment illustrated in FIG. 3 are arranged in a manner to more efficiently support kitchen labor and workflow, and are intended to centralize all of the ingredient control activities for packaging the food products for the precision temperature cooking system in one station. When the cooked/stored food products are desired for food service, they may be withdrawn from refrigerated storage 248 and placed in the heated water bath 230 to regenerate the food product to the desired temperature, and then removed from the vacuum sealable bags on the work surface 220 and transferred directly to the finishing devices at other location(s) in the system for finishing the surface of the food product, and then placed in the warmer drawer 260 (as needed) until required for food service delivery. Alternatively, when chilled storage of the cooked food products is not required, the food products may be removed from the heated water baths 230 and removed from their bags on the work surface 220, and then transferred directly to a finishing device for finishing the surface of the food product and then temporarily stored in warmer drawers 260, or delivered directly for food service.

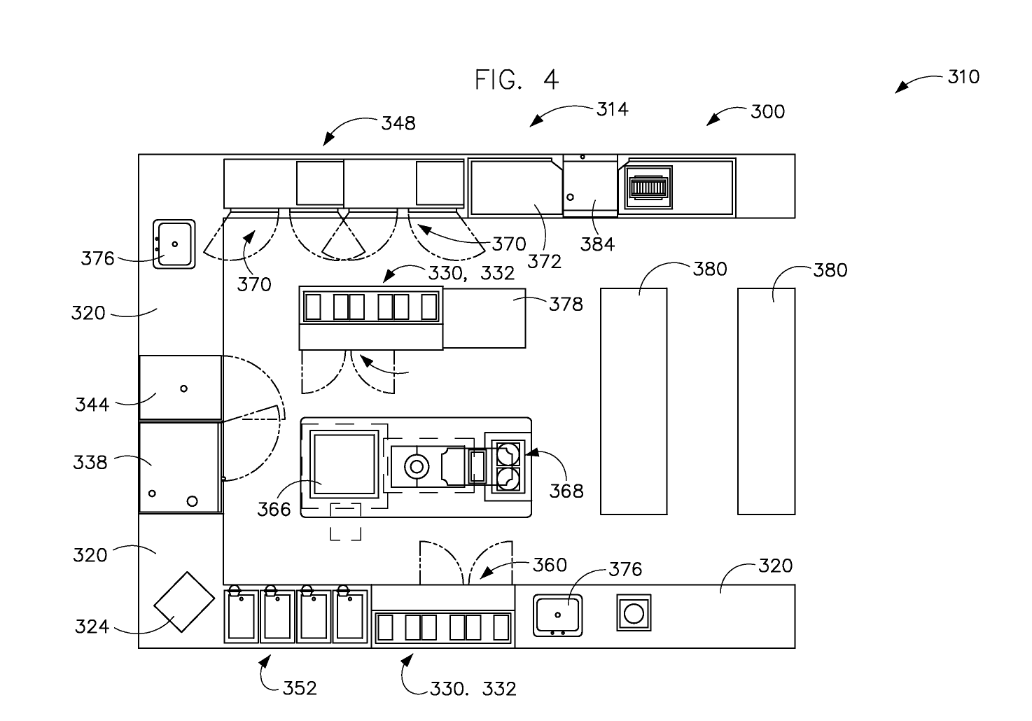

Referring now to FIG. 4, a precision temperature cooking system 300 is shown according to another exemplary embodiment to be included within a precision temperature kitchen 310 of a facility. System 300 is shown to include a combination of precision temperature cooking components packaged in a precision temperature cooking suite 314. Suite 314 comprises an enclosure intended to hold all of the components in space-saving, self-contained orientation, and in a sequence that enhances workflow of the precision temperature cooking process. Suite 314 may be formed from any suitable material intended to withstand the rigors of a high-demand kitchen environment, such as stainless steel, and may be internally pre-wired and pre-plumbed to all components to provide a single external plumbing and wiring connection to facilitate installation at a facility. Suite 314 also includes a work surface 320 that may include any one of a wide variety of suitable materials, such as a solid surface material (e.g. Corian®, Avonite®, etc.), or a wood material such as a maple or the like, stainless steel, or other suitable material.

The precision temperature cooking components are shown to include a vacuum packaging machine 324, heated water bathes 330 and heated water circulators 332, a combi-oven 338, a blast-chiller 344, a refrigerated storage compartment 348, a finishing device 352 shown as a four-zone plancha, and a warmer cabinet 360. Other components of the precision temperature kitchen are shown to include a “salamander” broiler 366, a radiant/induction cooktop 368 (e.g. “hob”, etc.), refrigerated storage devices (shown as an under-counter refrigerator 370 and a reach-in refrigerator 372), preparation sinks 376, preparation rails 378, chefs tables 380, and dishwashing device 384.

The precision temperature cooking components according to the exemplary embodiment illustrated in FIG. 4 are intended to integrate and coordinate with other components often used in a full-service kitchen, in a manner that supports preparation of the food products for precision temperature cooking (e.g. washing, cutting, seasoning, vacuum packaging, etc.) and then supports blast-chilling and holding the food products (as needed) until desired for food service and then the food products are regenerated and finished on an appropriate high temperature finishing component (e.g. combi-oven, salamander, induction hob, etc.). The remaining components of the precision temperature kitchen are intended to support other operations of the precision temperature kitchen, such as cleaning the components and serving the food products. The components of the precision temperature kitchen are arranged in a manner to more efficiently support kitchen labor and workflow. For example, according to the illustrated embodiment, the components are arranged in the following manner: a finishing station having suitable high temperature components and warming drawers and the like may be provided in a middle or “island” type of arrangement and having suitable ventilation (e.g. down-draft, overhead hood, etc.), and the other components of the system may be arranged to at least partially surround the finishing station (e.g. in a U or L shape, or semi-circular manner—depending on the space available in the kitchen). For example, a cleaning and preparation station (e.g. for unprepared food products and the like-with suitable sinks, work surfaces, etc.) may be provided at one end, followed by a vacuum packaging station, followed by a precision-temperature cooking station having suitable water baths and combi-ovens, followed by another station having a work surface for handling the prepared (e.g. cooked, etc.) food products, followed by a blast-chilling station having suitable flash-freezing devices, and followed by a controlled temperature storage station (e.g. walk-in coolers or the like) for maintaining the cooked and flash-frozen food products for later rethermalizing and finishing). However, the various stations of the precision temperature cooking system may be arranged in any desired manner to fit the available space of the kitchen or other installation and may include more, or less, components and equipment as necessary to provide a customized system intended to suit a particular application and/or type of food products to be prepared.

Accordingly, a precision temperature cooking system may include any one or more of the precision temperature cooking components arranged in any desired configuration intended to facilitate a workflow in a particular facility and type of food products to be served. Further, a precision temperature kitchen may include one or more precision temperature cooking suites, arranged with (or integrated among) other kitchen components to permit a facility to transition from a “high temperature” method of cooking to a “precision temperature” method of cooking.

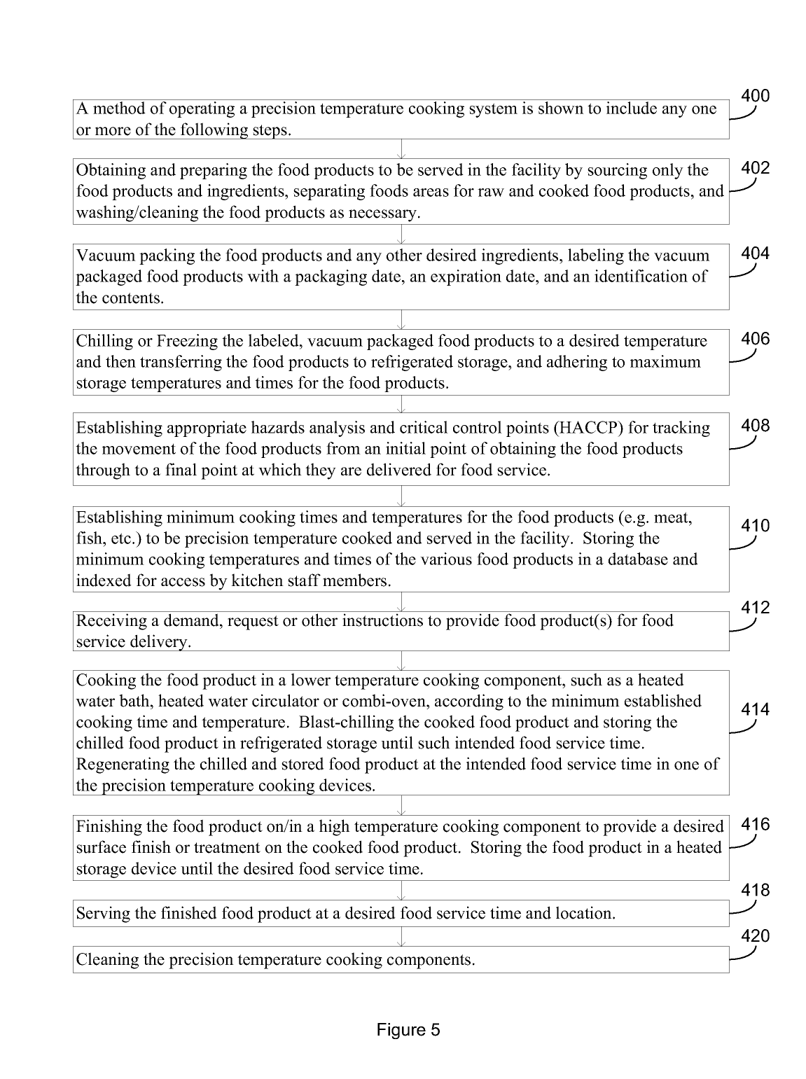

Referring now to FIG. 5, a method 400 of operating a precision temperature cooking system is shown according to an exemplary embodiment. The method of operating a precision temperature cooking system is shown to include any one or more of the following steps.

The step 402 includes obtaining and preparing the food products to be served in the facility by sourcing only the highest quality food products and ingredients, providing separate foods areas (e.g. areas on the work surface) for raw food products and cooked food products, and washing/cleaning the food products as necessary.

The step 404 includes vacuum packing the food products and any other desired ingredients (e.g. seasonings, spices, sauces, etc.) using at least two vacuum packaging machines (one for raw food products and one for cooked food products) labeling the vacuum packaged food products with a packaging date, an expiration date, and an identification of the contents (e.g. food product, type of other ingredients, dietary restriction notations, intended food service event, etc.). This step may be performed at a designated ingredient control station (as previously described with reference to FIG. 3) or any other suitable portion of the precision temperature cooking system or kitchen.

The step 406 includes blast-chilling the labeled, vacuum packaged food products to a desired temperature and then transferring the chilled, labeled, vacuum packaged food products to refrigerated storage to establish a prepared food product ready to be cooked (or regenerated if the food product has already been already cooked), and adhering to maximum storage temperatures and times for the prepared food products.

The step 408 includes establishing appropriate hazards analysis and critical control points (HACCP) for tracking the movement of the food products from an initial point of obtaining the food products through to a final point at which they are delivered for food service. Such control points may include (among others) verifying the quality of water in the kitchen, identifying potential breaks in the “cold chain” (i.e. the duration from the time that the food product is first chilled until it is removed for cooking and/or regenerating. Step 408 also includes identifying any unnecessary holding times, and identifying any potential contamination points with other food products or items in the kitchen, and monitoring for improper food product stock identification and rotation, and monitoring the time taken to cool any cooked food products for storage.

The step 410 includes establishing minimum cooking times and temperatures for the food products (e.g. meat, fish, etc.) to be precision temperature cooked and served in the facility. According to one embodiment, the cooking temperature should be greater than about 65° C. to ensure pasteurization, and the cooking time should be of sufficient duration to ensure that the core temperature of the food product has been reached (accounting for any initial temperature transient of the heated water bath upon introduction of chilled food products). Lower cooking temperatures may be used for certain food products, however, such food products should be consumed directly after cooking to minimize risks associated with possible bacterial contamination of the food product. According to one embodiment, the cooking temperature should not exceed about 70° C. to ensure that the moisture and flavor is not reduced to an undesirable degree. According to other embodiments, the cooking temperatures may be substantially within the range of 50° C.-90° C. According to one embodiment, the cooking times and temperatures for various food products are established during a trail stage by using an accessory probe placed in the food product, a digital thermometer to determine when the desired temperature of the core of the food product has been reached, and a timing device (e.g. timer, stop-watch, etc.) to establish the time duration necessary for the core of a particular food product to be reached when cooked at a particular temperature. Such a trial stage may be conducted for all food products intended to be served by the facility, beginning with a cooking temperature that is approximately equal to the desired finished core temperature of the cooked food product. Upon any changes to the scope of food products served by the facility (e.g. menu changes, specials, substitutions, revised dietary restrictions/specifications for particular groups or individuals, etc.), a new trial stage for the new/changed food products may be conducted. The minimum cooking temperatures and times of the various food products are stored in a database (e.g. an appropriate computer device or the like such as the computing device and user interface 250 as previously described with reference to FIG. 3) and indexed, arranged or sorted by food product type or the like (e.g. alphabetically or by other suitable method) so that the cooking temperature and time data may be quickly and conveniently recalled or consulted by kitchen staff members (e.g. using appropriate icons representative of particular food types, “drop-down” menus or the like, etc.).

The step 412 includes receiving a demand, request or other instructions to provide food product(s) for food service delivery. Such a demand may be based on a “regular frequency” type event (e.g. regularly scheduled food service times and events for large institutional facilities based on a previously established menu), or the request may be individually based during certain predefined time periods (e.g. menu-ordered from diners in restaurants and the like), or may be individually based at any time (e.g. room-service orders in hotels), or the instructions may be individually based on special dietary requirements established for individuals (or groups), such as may be desired in health facilities or assisted care living facilities, etc., such instructions may be generated from within the facility, or may be received (e.g. by computer, etc.) from a remote location (such as those intended to coordinate food service for multiple facilities or the like).

The step 414 includes cooking the food product in a lower temperature cooking component, such as a heated water bath, heated water circulator or combi-oven, according to the minimum established cooking time. If the cooked food product is intended for storage until a later food service time, then blast-chilling the cooked food product and storing the chilled food product in refrigerated storage until such intended food service time. A next step may include regenerating the chilled and stored food product at the intended food service time in one of the precision temperature cooking devices.

Following regeneration (or cooking if the cooked food product is intended for direct food service), a next step 416 may include finishing the food product on/in a high temperature cooking component (e.g. plancha, griddle, grill, burner, barbecue, rotisserie, oven, deep fryer, etc.) to provide a desired surface finish or treatment on the cooked food product. In the event that a large volume of food products are being served for a single food service event (e.g. banquet, etc), a next step may include storing the food product in a heated storage device (e.g. warmer drawer, etc.) until the food product is ready to be served. It should be noted that the food products may be finished before or after heated storage, as desired by a particular chef for a particular food product.

The step 418 includes serving the finished food product at a desired food service time and location.

The step 420 includes cleaning the precision temperature cooking components. According to an exemplary embodiment, the system is intended to result in a reduction of cleaning activities (in comparison to conventional high temperature kitchens and cooking systems) by reducing the number of pots, pans, containers, utensils and the like needed to process the food, because the food products may be cooked in advance using a relatively small number of precision temperature cooking components, which contain water and are readily and easily cleaned.

Accordingly, a method of operating a precision temperature cooking system may include any one or more of the foregoing steps, and in any sequence or order desired to accommodate a particular facility and type of food products to be served.

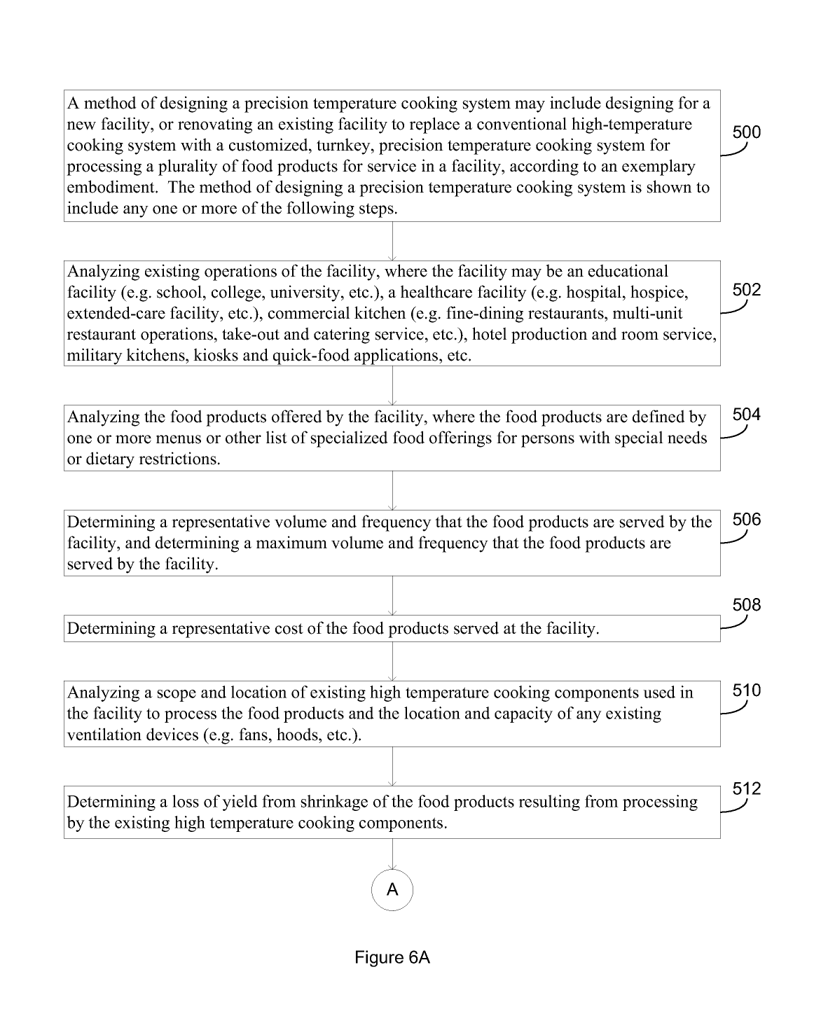

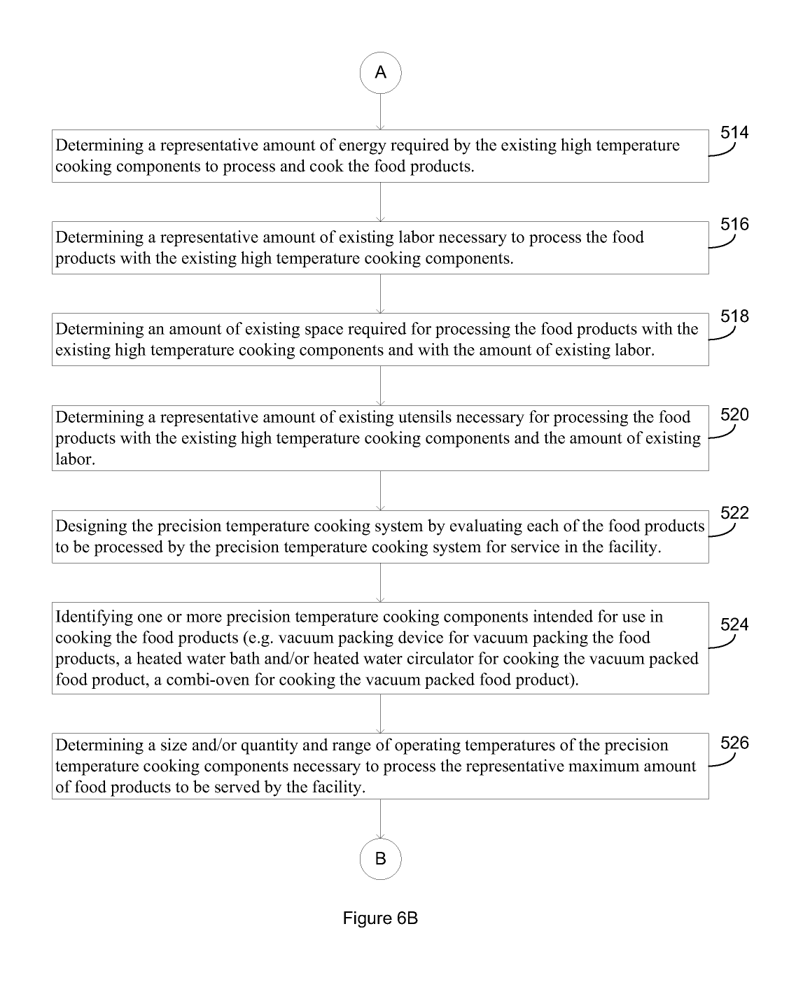

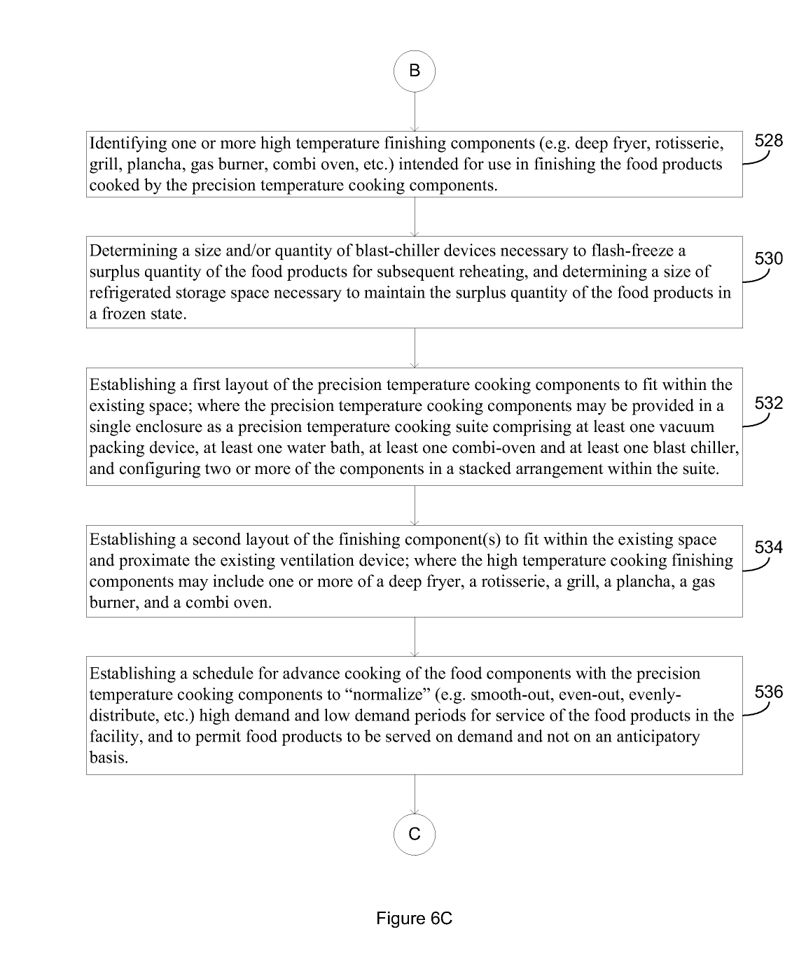

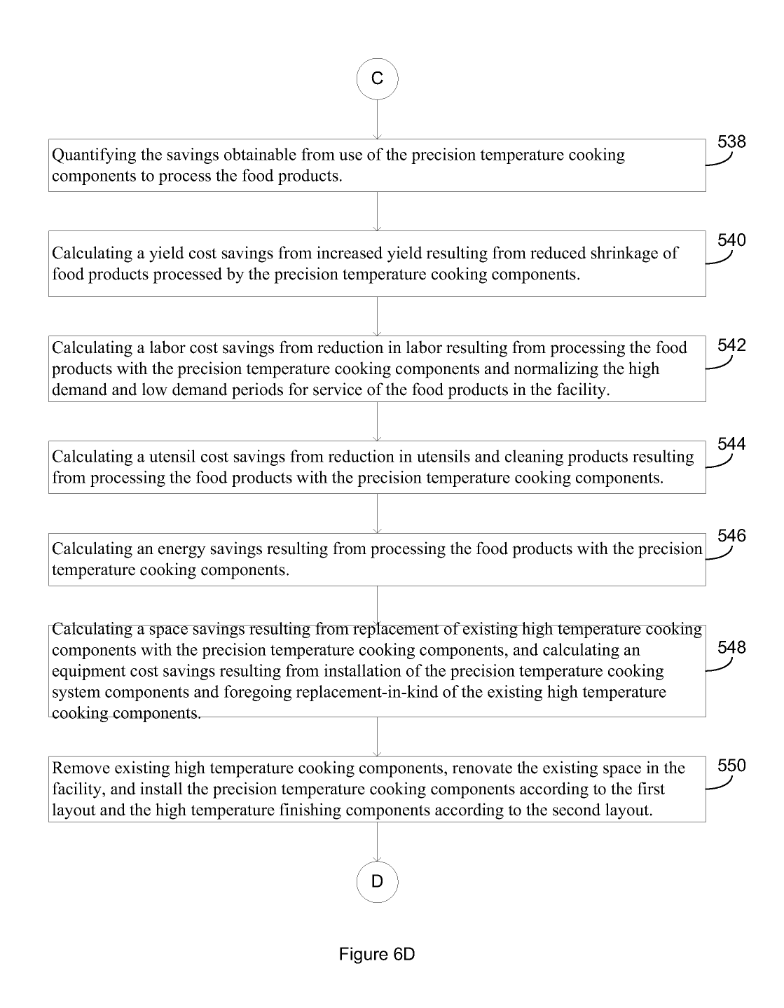

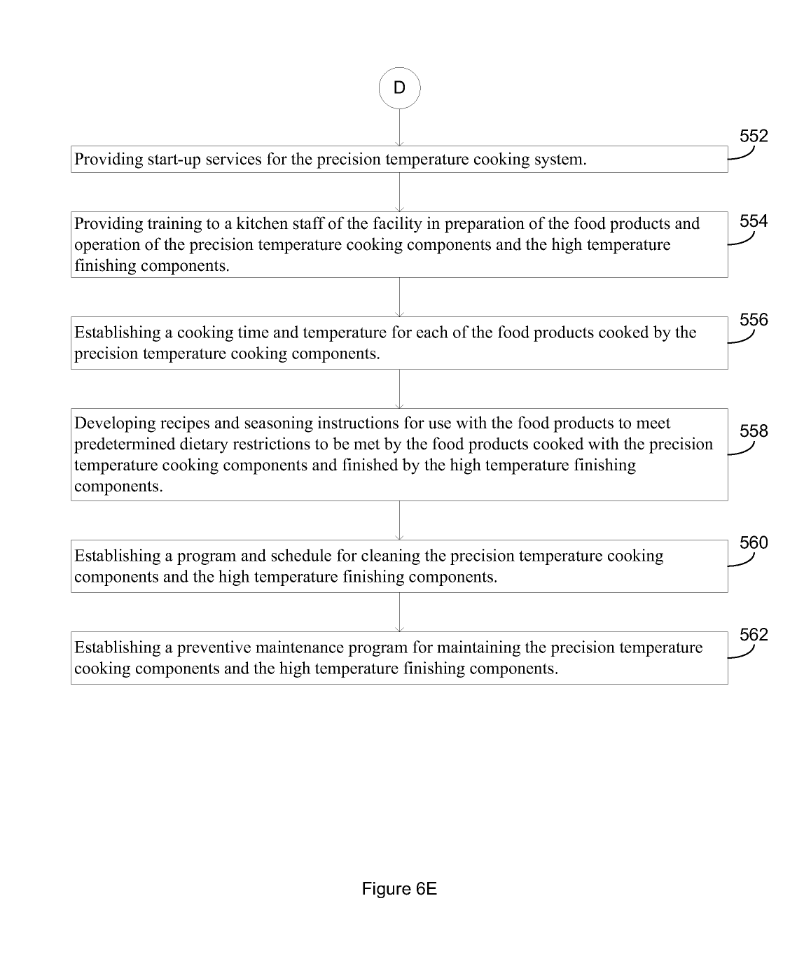

Referring now to FIGS. 6A-6E, a method 500 of designing a precision temperature cooking system may include designing for a new facility, or renovating an existing facility to replace a conventional high-temperature cooking system with a customized, turnkey, precision temperature cooking system for processing a plurality of food products for service in a facility, according to an exemplary embodiment. The method 500 of designing a precision temperature cooking system is shown to include any one or more of the following steps.

The step 502 includes analyzing existing operations of the facility, where the facility may be an educational facility (e.g. school, college, university, etc.), a healthcare facility (e.g. hospital, hospice, extended-care facility, etc.), commercial kitchen (e.g. fine-dining restaurants, multi-unit restaurant operations, take-out and catering service, etc.), hotel production and room service, military kitchens, kiosks and quick-food applications, etc.

The step 504 includes analyzing the food products offered by the facility, where the food products are defined by one or more menus or other list of specialized food offerings for persons with special needs or dietary restrictions.

The step 506 includes determining a representative volume and frequency that the food products are served by the facility, and determining a maximum volume and frequency that the food products are served by the facility.

The step 508 includes determining a representative cost of the food products served at the facility.

The step 510 includes analyzing a scope and location of existing high temperature cooking components used in the facility to process the food products and the location and capacity of any existing ventilation devices (e.g. fans, hoods, etc.).

The step 512 includes determining a loss of yield from shrinkage of the food products resulting from processing by the existing high temperature cooking components.

The step 514 includes determining a representative amount of energy required by the existing high temperature cooking components to process and cook the food products.

The step 516 includes determining a representative amount of existing labor necessary to process the food products with the existing high temperature cooking components.

The step 518 includes determining an amount of existing space required for processing the food products with the existing high temperature cooking components and with the amount of existing labor.

The step 520 includes determining a representative amount of existing utensils necessary for processing the food products with the existing high temperature cooking components and the amount of existing labor.

The step 522 includes designing the precision temperature cooking system by evaluating each of the food products to be processed by the precision temperature cooking system for service in the facility.

The step 524 includes identifying one or more precision temperature cooking components intended for use in cooking the food products (e.g. vacuum packing device for vacuum packing the food products, a heated water bath and/or heated water circulator for cooking the vacuum packed food product, a combi-oven for cooking the vacuum packed food product).

The step 526 includes determining a size and/or quantity and range of operating temperatures of the precision temperature cooking components necessary to process the representative maximum amount of food products to be served by the facility.

The step 528 includes identifying one or more high temperature finishing components (e.g. deep fryer, rotisserie, grill, plancha, gas burner, combi oven, etc.) intended for use in finishing the food products cooked by the precision temperature cooking components.

The step 530 includes determining a size and/or quantity of blast-chiller devices necessary to blast-chill or flash-freeze a surplus quantity of the food products for subsequent reheating, and determining a size of refrigerated storage space necessary to maintain the surplus quantity of the food products in a frozen state.

The step 532 includes establishing a first layout of the precision temperature cooking components to fit within the existing space; where the precision temperature cooking components may be provided in a single enclosure as a precision temperature cooking suite comprising at least one vacuum packing device, at least one water bath, at least one combi-oven and at least one blast chiller, and configuring two or more of the components in a stacked arrangement within the suite.

The step 534 includes establishing a second layout of the finishing component(s) to fit within the existing space and proximate the existing ventilation device; where the high temperature cooking finishing components may include one or more of a deep fryer, a rotisserie, a grill, a plancha, a gas burner, and a combi oven.

The step 536 includes establishing a schedule for advance cooking of the food components with the precision temperature cooking components to “normalize” (e.g. smooth-out, even-out, evenly-distribute, etc.) high demand and low demand periods for service of the food products in the facility, and to permit food products to be served on demand and not on an anticipatory basis.

The step 538 includes quantifying the savings obtainable from use of the precision temperature cooking components to process the food products by (540) calculating a yield cost savings from increased yield resulting from reduced shrinkage of food products processed by the precision temperature cooking components, and (542) calculating a labor cost savings from reduction in labor resulting from processing the food products with the precision temperature cooking components and normalizing the high demand and low demand periods for service of the food products in the facility, and (544) calculating a utensil cost savings from reduction in utensils and cleaning products resulting from processing the food products with the precision temperature cooking components, and (546) calculating an energy savings resulting from processing the food products with the precision temperature cooking components, and (548) calculating a space savings resulting from replacement of existing high temperature cooking components with the precision temperature cooking components, and calculating an equipment cost savings resulting from installation of the precision temperature cooking system components and foregoing replacement-in-kind of the existing high temperature cooking components.

The step 550 includes removing existing high temperature cooking components, renovating the existing space in the facility, and installing the precision temperature cooking components according to the first layout and the high temperature finishing components according to the second layout.

The step 552 includes providing start-up services for the precision temperature cooking system, including (554) providing training to a kitchen staff of the facility in preparation of the food products and operation of the precision temperature cooking components and the high temperature finishing components, (556) establishing a cooking time and temperature for each of the food products cooked by the precision temperature cooking components, (558) developing recipes and seasoning instructions for use with the food products to meet predetermined dietary restrictions to be met by the food products cooked with the precision temperature cooking components and finished by the high temperature finishing components, (560) establishing a program and schedule for cleaning the precision temperature cooking components and the high temperature finishing components, (562) establishing a preventive maintenance program for maintaining the precision temperature cooking components and the high temperature finishing components.

Accordingly, a method of providing a precision temperature cooking system (for a new facility, or to renovate an existing “high temperature” facility) may include any one or more of the foregoing steps, and in any sequence or order desired to accommodate a particular facility and type of food products to be served. Further, one or more of the steps involved in the method of providing a precision temperature cooking system may be implemented using software and any suitable computing device configured to receive inputs for the variable parameters of the system and to compute desired quantities or values. Further the computing device may include suitable database capabilities for storing and providing access to various data such as cooking times and temperatures, recipes, special dietary requirements, menus, food product volume and service frequency data, etc. Accordingly, the various steps of the method of providing a precision temperature kitchen may be machine-implemented using a computing device and suitable software according to any exemplary embodiment.

It is also important to note that the construction and arrangement of the elements of the precision temperature cooking system, the method of operating the precision temperature cooking system and the method of designing a precision temperature cooking system as shown in the preferred and other exemplary embodiments is illustrative only. Although only a few embodiments of the present inventions have been described in detail in this disclosure, those skilled in the art who review this disclosure will readily appreciate that many modifications are possible (e.g., variations in sequence, sizes, dimensions, structures, shapes, profiles and proportions of the various elements, values of parameters, mounting arrangements, use of materials, orientations, compositions of materials, etc.) without materially departing from the novel teachings and advantages of the subject matter recited. For example, elements shown as integrally formed may be constructed of multiple parts or elements show as multiple parts may be integrally formed. It should also be noted that the components of the precision temperature cooking system and the precision temperature kitchen may be used in association with a wide variety of applications (e.g. hospital and/or healthcare facilities, restaurants, private residences, hotels, private clubs, fast-food type applications, portable food vendors, banquet and catering operations, etc.) and that the elements and components of the system may be provided in any suitable size, shape, material and appearance that meets applicable design and performance standards. Accordingly, all such modifications are intended to be included within the scope of the present inventions. Other substitutions, modifications, changes and omissions may be made in the design, operating conditions and arrangement of the preferred and other exemplary embodiments without departing from the spirit of the present inventions.

The order or sequence of any process or method steps may be varied or re-sequenced according to alternative embodiments. Other substitutions, modifications, changes and omissions may be made in the design, operating configuration and arrangement of the preferred and other exemplary embodiments without departing from the spirit of the inventions as expressed in the appended claims.

We claim:

1. A precision temperature cooking system for food products, comprising:
a vacuum packaging device configured to package the food products in vacuum-sealable bags;
at least one of a heated water bath and a heated water circulator configured to maintain a water bath at a cooking temperature that is substantially equal to a desired final temperature of a core portion of the food products;
a chilling device configured to chill or freeze the food products cooked by the water bath;
a refrigerated storage device configured to receive and store the frozen food products from the chilling device; and
a high-temperature finishing component configured to impart a desired appearance to the outside of the food product.

2. The system of claim 1 wherein the vacuum packaging device and the heated water bath and the chilling device and the refrigerated storage device and the high temperature finishing component are packaged into a single enclosure to form a precision temperature cooking suite.

3. The system of claim 1 wherein the water bath is also operable to regenerate the food products to the desired final temperature following removal of the food products from refrigerated storage device.

4. The system of claim 3 wherein the cooking temperature and the desired final temperature are substantially within a range of 50° C. to 90° C.

5. The system of claim 4 wherein the high temperature finishing component is selected from the group consisting of a plancha, a griddle, a grill, an oven, a combi-oven, a rotisserie, a broiler, and a deep fryer.

6. The system of claim 1 further comprising an ingredient control center, the ingredient control center including a user interface device configured to display ingredients for packaging with the food products.

7. A method of operating a precision temperature cooking system for cooking food products in a facility, comprising:
identifying a scope of food products to be served by the facility;
obtaining and preparing the food products to be served in the facility by sourcing the food products, providing separate areas on a work surface for raw food products and cooked food products, and for cleaning the food products;
vacuum packing the food products with other ingredients, labeling the food products with a packaging date, an expiration date, and an identification of the food product;
blast-chilling the food products to a desired storage temperature and then transferring the food products to a refrigerated storage device;
establishing minimum cooking times and minimum cooking temperatures for the food products;
storing the minimum cooking times and minimum cooking temperatures in a database;
cooking the food products in a precision temperature cooking component operating at a temperature substantially equal to a desired final temperature of a core portion of the food product;
receiving instructions to provide the food products for food service delivery;
regenerating the food product in the lower temperature cooking component; and
finishing the food product on a high temperature cooking component to provide a desired surface condition on the food product.

8. The method of claim 7 further comprising the step of establishing hazards analysis and critical control points for tracking movement of the food products from an initial point of obtaining the food products through to a final point at which the food products are delivered for food service.

9. The method of claim 7 wherein the precision temperature cooking component comprises at least one of a heated water bath, heated water circulator or combi-oven.

10. The method of claim 7 wherein the step of receiving instructions comprises receiving dietary information for the food product to be prepared for a particular consumer having dietary restrictions.

11. The method of claim 7 wherein the step of establishing minimum cooking times and minimum cooking temperatures further comprises conducting a trial stage using a probe placed in the food product, a digital thermometer to determine when the final desired temperature of the core portion of the food product has been reached, and a timing device to establish a duration for the core portion of the food product to reach the final desired temperature.

12. A method of providing a precision temperature cooking system for cooking food products in a facility, comprising:
analyzing existing operations of the facility, comprising the steps of:
analyzing the food products offered by the facility, where the food products are defined by one or more menus;
determining at least one of a representative volume and frequency that the food products are served by the facility, and a maximum volume and frequency that the food products are served by the facility;
analyzing a scope and location of existing high temperature cooking components used in the facility to cook the food products and a location and a capacity of any existing ventilation devices;
determining a representative amount of energy required by the existing high temperature cooking components to cook the food products;
determining a representative amount of existing labor necessary to cook the food products with the existing high temperature cooking components;
determining an amount of existing space required for processing the food products with the existing high temperature cooking components and with the amount of existing labor;
designing the precision temperature cooking system by evaluating the food products to be served in the facility, comprising the steps of:
identifying one or more precision temperature cooking components for cooking the food products;
determining a size and a quantity of the precision temperature cooking components necessary to cook a quantity of food products sufficient to meet the maximum volume and frequency that the food products are served by the facility;
identifying one or more high temperature finishing components for use in finishing the food products cooked by the precision temperature cooking components;
determining a size and a quantity of chiller-freezer devices sufficient to chill or flash-freeze a surplus quantity of the food products for subsequent regenerating;
determining a size of refrigerated storage space sufficient to maintain the surplus quantity of the food products in a frozen state;
establishing a first layout of the precision temperature cooking components to fit within the existing space;
establishing a second layout of the finishing components to fit within the existing space and proximate the existing ventilation device.

13. The method of claim 12, further comprising quantifying a savings obtainable from use of the precision temperature cooking components to cook the food products, including:
calculating a labor cost savings from reduction in labor resulting from cooking the food products with the precision temperature cooking components and normalizing high demand periods and low demand periods for service of the food products in the facility;
calculating an energy savings resulting from cooking the food products with the precision temperature cooking components.

14. The method of claim 12, wherein the facility is one of an educational facility, a healthcare facility, a fine-dining restaurant, a multi-unit restaurant, a take-out service, a catering service, a hotel, a military kitchens, a kiosk and a quick-food restaurant.

15. The method of claim 12 further comprising determining a loss of yield from shrinkage of the food products resulting from cooking with the existing high temperature cooking components.

16. The method of claim 12 further comprising determining a representative amount of existing utensils necessary for processing the food products with the existing high temperature cooking components and the amount of existing labor, and calculating a utensil cost savings from reduction in utensils and cleaning products resulting from cooking the food products with the precision temperature cooking components.

17. The method of claim 12 wherein the precision temperature cooking components comprise at least one of a heated water bath and a heated water circulator and a combi-oven.

18. The method of claim 12 where the precision temperature cooking components are provided in a single enclosure as a precision temperature cooking suite that also includes at least one vacuum packing device, at least one chiller-freezer device.

19. The method of claim 12 wherein the high temperature finishing component is selected from the group consisting of a deep fryer, a rotisserie, a grill, a plancha, a gas burner, a combi oven, a griddle, and oven and a broiler.

20. The method of claim 12 further comprising establishing a schedule for advance cooking of the food components with the precision temperature cooking components to normalize high demand and low demand periods for service of the food products in the facility.

21. The method of claim 12 further comprising calculating a space savings resulting from replacement of the existing high temperature cooking components with the precision temperature cooking components, and calculating an equipment cost savings resulting from installation of the precision temperature cooking system components and foregoing replacement-in-kind of the existing high temperature cooking components.

22. The method of claim 12 further comprising removing the existing high temperature cooking components and installing the precision temperature cooking components according to the first layout and the high temperature finishing components according to the second layout.

23. The method of claim 12 further comprising the step of providing start-up services for the precision temperature cooking system, which comprises one or more of the steps of:
providing training to a kitchen staff of the facility in preparation of the food products and operation of the precision temperature cooking components and the high temperature finishing components;
establishing a cooking time and temperature for each of the food products cooked by the precision temperature cooking components;
developing recipes and seasoning instructions for use with the food products to meet predetermined dietary restrictions for the food products cooked with the precision temperature cooking components and finished by the high temperature finishing components;
establishing a program and schedule for cleaning the precision temperature cooking components and the high temperature finishing components; and
establishing a preventive maintenance program for maintaining the precision temperature cooking components and the high temperature finishing components.

24. The method of claim 12 further comprising determining a representative cost of the food products served at the facility.

25. The method of claim 24 further comprising the step of calculating a yield cost savings from increased yield resulting from reduced shrinkage of food products cooked by the precision temperature cooking components and the representative cost of the food products served at the facility.
